(19) United States
(12) Patent Application Publication
(10) Pub. No.: US 2020/0032188 A1
(43) Pub. Date: Jan. 30, 2020

(54) CULTURE MEMBRANE AND CULTURE DISH

(71) Applicant: AUROTEK CORPORATION, Taipei (TW)

(72) Inventors: Wen-Shan Chou, Taipei (TW); Chamin Tien, Taipei (TW)

(21) Appl. No.: 16/522,666

(22) Filed: Jul. 26, 2019

(30) Foreign Application Priority Data
Jul. 27, 2018 (TW) 107126168

(51) Int. Cl. C12M 1/12; C12M 1/00

(52) U.S. Cl. CPC C12M 25/04; C12M 23/06; C12M 23/20

(57) ABSTRACT

A culture membrane having a first surface is provided. The first surface has a plurality of protrusions. The tops of the protrusions and the gaps between the tops jointly form a culture portion adapted to culture cells. The culture portion has a hardness of 2 kPa to 500 kPa. The invention further provides a culture dish. The culture membrane of the invention and the culture dish using the same may increase the survival rate of cells.

### FIELD OF THE INVENTION

The present invention relates to a culture medium, and more particularly to a culture membrane and a culture dish using the same.

### BACKGROUND OF THE INVENTION

In today's society with advanced medical technology, there are still many diseases that cannot be completely cured. Cancer is one of the examples. Many scientists are currently working to identify factors that turn into cancer cells. Experimental research on cancer cells has made it difficult to make breakthroughs in recent years. One of the reasons is that cancer cells are difficult to culture in vitro.

Cell culture is a biotechnology that refers to the cultivation of biological cells in a controlled state to grow them. The development and methods of the technology are closely related to tissue culture or organ culture. However, compared to general cells, little research has been done on the culture techniques of cancer cells, so many uncertainties are generated during the culture process. The current thinking for cancer cell culture is to enable cancer cells to grow in a culture environment that simulates the environment of the human body.

Conventional culture dish materials are mostly plastic or glass materials, which have the advantages of being cheap and easy to obtain. However, since the conventional culture dish cannot simulate the environment of the human body, it is difficult for cancer cells to grow.

### SUMMARY OF THE INVENTION

The present invention provides a culture membrane which may increase the survival rate of cells.

The present invention provides a culture dish which may increase the survival rate of cells.

A culture membrane provided in an embodiment of the invention has a first surface. The first surface has a plurality of protrusions. The tops of the plurality of protrusions and the gaps between the tops jointly form a culture portion adapted to culture cells. The culture portion has a hardness of 2 kPa to 500 kPa.

In one embodiment of the invention, the culture portion has a hardness of 2 kPa to 20 kPa.

In one embodiment of the invention, a shape of the plurality of protrusions includes a conical shape, a cylindrical shape or a truncated cone shape.

In one embodiment of the invention, a spacing between centers of the tops of the plurality of protrusions is less than or equal to half a size of a cell.

In one embodiment of the invention, a material of the culture membrane has a Shore hardness of less than 60 HA, and an area of an orthographic projection of the tops of the plurality of protrusions on the first surface as a percentage of an area of the first surface is 20% to 70%.

In one embodiment of the invention, a distance between centers of the tops of the plurality of protrusions is 0.4 μm to 1 μm.

In one embodiment of the invention, a material of the culture membrane includes silicone.

In one embodiment of the invention, a material of the culture membrane has a Shore hardness of more than 60 HA, and an area of an orthographic projection of the tops of the plurality of protrusions on the first surface as a percentage of an area of the first surface is 0.01% to 0.001%.

In one embodiment of the invention, a distance between centers of the tops of the plurality of protrusions is 1 μm to 3 μm.

In one embodiment of the invention, a material of the culture membrane includes sapphire.

In one embodiment of the invention, the culture membrane further has a second surface opposite to the first surface, and the first surface and the second surface are both polished surfaces.

A culture dish provided in an embodiment of the invention includes a dish body and the culture membrane. The culture membrane is disposed on a bottom surface of the dish body.

The culture membrane of the invention has a culture portion. Since the culture portion has a hardness of 2 kPa to 500 kPa, which overlaps with the hardness range of the human tissue, the in vivo environment of cell growth may be simulated, and the survival rate of the cultured cells may increase. The culture dish of the invention uses the culture membrane, and therefore has the same advantages.

Other objectives, features and advantages of The invention will be further understood from the further technological features disclosed by the embodiments of The invention wherein there are shown and described preferred embodiments of this invention, simply by way of illustration of modes best suited to carry out the invention.

### BRIEF DESCRIPTION OF THE DRAWINGS

The present invention will become more readily apparent to those ordinarily skilled in the art after reviewing the following detailed description and accompanying drawings, in which:

FIG. 1 is a schematic diagram of a culture membrane of one embodiment of the invention;

FIG. 2 is a schematic cross-sectional diagram along a line AA of FIG. 1;

FIG. 3A to FIG. 3D are explanatory diagrams for adjusting a hardness of a culture portion;

FIG. 4A is a schematic diagram of an image of cells cultured in a conventional culture dish under a microscope;

FIG. 4B is a schematic diagram of an image of cells cultured in a culture membrane of one embodiment of the invention under a microscope;

FIG. 5 is a schematic diagram of different shapes of protrusions of a first surface;

FIG. 6 is a schematic diagram of cultured cells of a culture membrane of one embodiment of the invention;

FIG. 7 is a schematic diagram of a culture membrane of another embodiment of the invention;

FIG. 8 is a schematic cross-sectional diagram of a culture membrane of another embodiment of the invention; and

FIG. 9 is a schematic perspective diagram of a culture dish of one embodiment of the invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

In the specification, the range represented by “one value to another value” is a schematic representation that avoids enumerating all the values in the range in the specification. Therefore, the recitation of a particular range of values is equivalent to the disclosure of any value in the range of values and a smaller range of values defined by any value within the range of values, as if the stated value and the smaller range of values are written in the specification. For example, the description of “the size is 10 mm to 100 mm” is equivalent to the disclosure of “the size is 20 mm to 50 mm”, regardless of whether other values are listed in the specification.

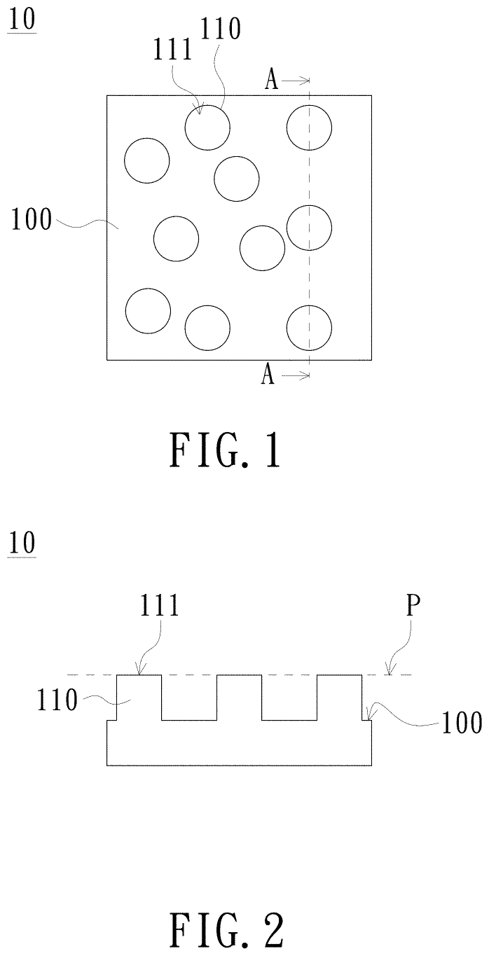

FIG. 1 is a schematic diagram of a culture membrane of one embodiment of the invention. FIG. 2 is a schematic cross-sectional diagram along a line AA of FIG. 1. Referring to FIG. 1 and FIG. 2, a culture membrane 10 of the embodiment has a first surface 100. The first surface 100 has a plurality of protrusions 110. The plurality of protrusions 110 may, for example, be evenly distributed or unevenly distributed over the first surface 100. The tops 111 of the plurality of protrusions 110 together form the culture portion P (shown by the dotted line in FIG. 2). Since the culture portion P further includes the gaps around the protrusions 110 in addition to the protrusions 110, the culture portion P is not a complete plane, but is not limited thereto. It should be noted that, in FIG. 2, for the convenience of labeling, the length of the dotted line is larger than the culture membrane 10, and is not shown as the area of the culture portion P. This representation also applies to the following figures. An area of an orthographic projection of the culture portion P on the first surface 100 is, for example, equal to or smaller than an area of the first surface 100. The culture portion P is adapted to culture cells, and has a hardness of 2 kPa to 500 kPa. When the culture membrane 10 is used to culture other human cell types other than human stem cells, the hardness of the culture portion P is preferably 2 kPa to 20 kPa. The relationship between the hardness of the culture portion P and the cell culture will be described in detail below.

Specifically, the hardness measurement of the culture portion P is performed by a device emitting ultrasonic waves, using the principle that the ultrasonic waves pass through the hard material faster, and the ultrasonic waves pass through the soft material at a slower speed to quantify the degree of hardness. The same method has been used in the human body as a medical test, such as liver cirrhosis test. The hardness may be regarded as the tissue stiffness. Through the device detection, the hardness of the organ tissues in different parts of the human body can be known, and the growth environment suitable for different types of human cells may be quantified as a numerical value.

The hardness range of the culture portion P of the invention is exemplified by the organ tissue of different parts of the human body, that is, the culture portion P is adapted to culture various cells of the human body, but is not limited thereto. The culture membrane 10 may also culture cells of other organisms as long as the hardness range of the culture portion P is set to be the same as the growth environment of the cells of other organisms.

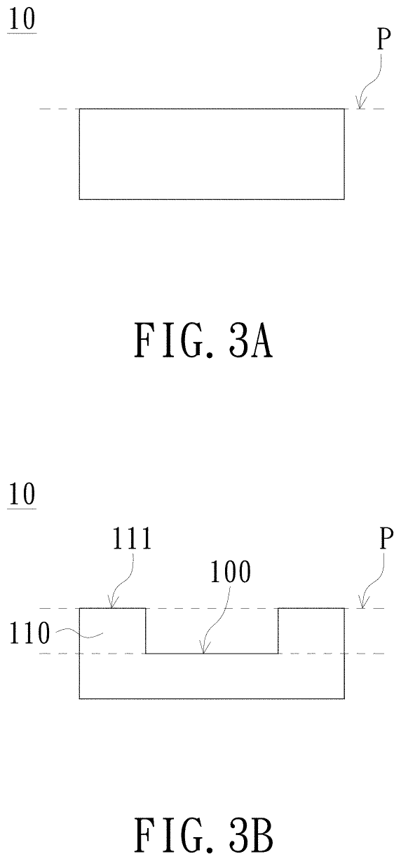

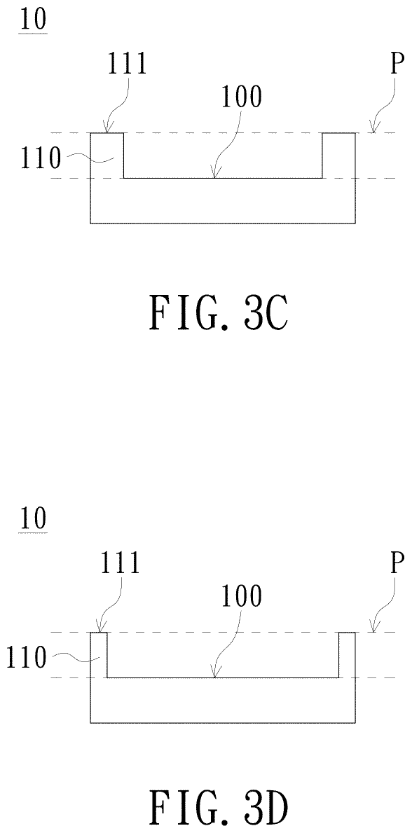

A value of the hardness of the culture portion P is adjusted by the area of the tops 111 and the quantity of the protrusions 110, which will be exemplified below. FIG. 3A to FIG. 3D are explanatory diagrams for adjusting a hardness of a culture portion. Referring to FIG. 3A to FIG. 3D, the culture membrane 10 in FIG. 3A does not have any protrusions, and the hardness of the culture portion P is assumed to be 10 kPa. An area of an orthographic projection of the tops 111 of the respective protrusions 110 in FIG. 3B on the first surface 100 as a percentage of an area of the first surface 100 is 50%, then the hardness of the culture portion P is 10 kPa×50%=5 kPa. In this way, an area of an orthographic projection of the tops 111 of the respective protrusions 110 in FIG. 3C on the first surface 100 as a percentage of an area of the first surface 100 is 25%, and the hardness of the culture portion P is 2.5 kPa. An area of an orthographic projection of the tops 111 of the respective protrusions 110 in FIG. 3D on the first surface 100 as a percentage of an area of the first surface 100 is 12.5%, and the hardness of the culture portion P is 1.25 kPa. To illustrate the definition of the hardness value, two protrusions 110 are exemplified in FIG. 3B to FIG. 3D, but the quantity of the protrusions 110 is not limited.

The culture membrane 10 of the invention has a culture portion P. Since the culture portion P has a hardness of 2 kPa to 500 kPa, which overlaps with the hardness range of the human tissue, the in vivo environment of cell growth may be simulated, and the survival rate of the cultured cells may increase.

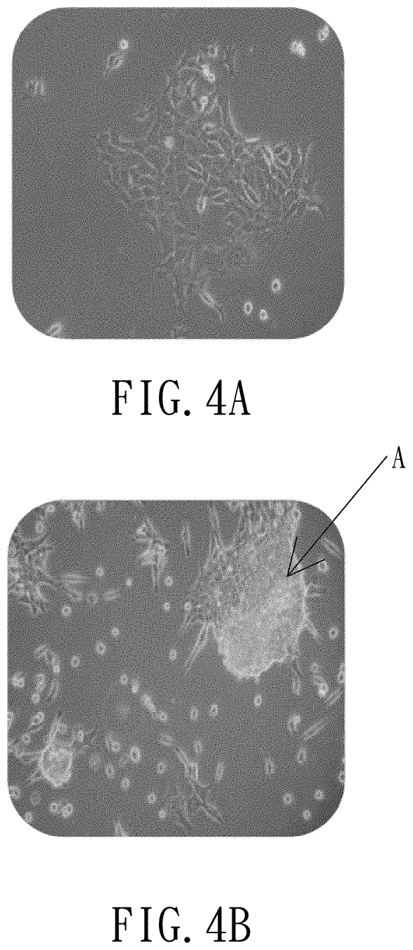

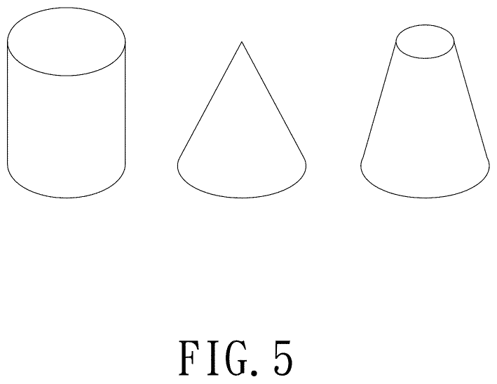

FIG. 4A is a schematic diagram of an image of cells cultured in a conventional culture dish under a microscope. FIG. 4B is a schematic diagram of an image of cells cultured in a culture membrane of one embodiment of the invention under a microscope. Referring to FIG. 4A and FIG. 4B, in the experiment, the culture of breast cancer cells is taken as an example. When cultured in a conventional culture dish (FIG. 4A), since the cell culture environment is not natural, the cells may be degenerated and the original characteristics may be changed, so that the survival rate of breast cancer cells is not high, or the growth is not good (the breast cancer cells are not found in FIG. 4A, and the cells in the figure are normal cells of breast tissue). When cultured in the culture membrane of the embodiment (FIG. 4B), since the hardness value of the culture portion is adjusted to be the same as that of the breast, it can be seen that the growth of the breast cancer cells (pointed by the arrow A) is good. For cell types that are currently more difficult to culture, such as cancer cells, if the survival rate of cultured cells is increased, subsequent research experiments may be made more advanced.

In the above figure, the plurality of protrusions is exemplified by a cylindrical shape, but is not limited thereto. FIG. 5 is a schematic diagram of different shapes of protrusions of a first surface. Referring to FIG. 5, the shape of the plurality of protrusions may also include a conical shape or a truncated cone shape. A cone with a region including its apex cut off by a plane is called a “truncated cone”. In addition, depending on the design requirements, the bottom surfaces of the plurality of protrusions are not limited to a circular shape, and the shape of the plurality of protrusions may also include, for example, a prism shape, a pyramid shape, or the like.

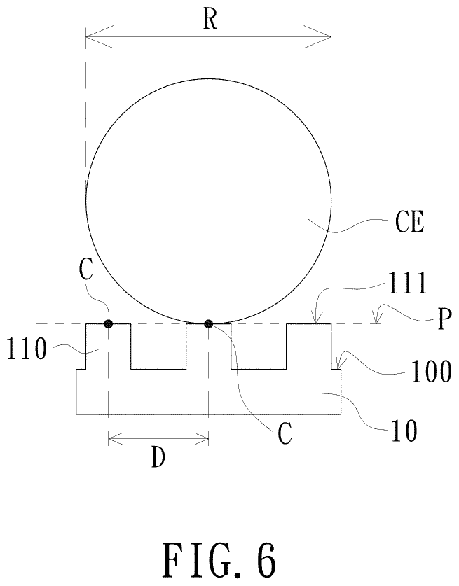

In order to achieve the effect of increasing the survival rate of the cultured cells, the cultured cells should be cultured in the culture portion P of the embodiment. FIG. 6 is a schematic diagram of cultured cells of a culture membrane of one embodiment of the invention. Referring to FIG. 6, a spacing D of the embodiment between the centers C of the tops 111 of the plurality of protrusions 110 should be less than or equal to half a size R of a cell CE, that is, the spacing D between the centers C of the tops 111 of the plurality of protrusions 110 should be adjusted according to the culture of different cell CE. For example, when the size R of the cell CE is 2 μm, the spacing D should be less than or equal to 1 μm. If the spacing D is greater than 1 μm, the cell CE may fall into depressions between the plurality of protrusions 110, even touching the first surface 100, causing a positional shift and not culture in the culture portion P, so the effect of increasing the survival rate of the cell CE may not be achieved. It should be noted that the cell CE in FIG. 6 is exemplified by a spherical shape, but is not limited thereto. Although the cell CE has a three-dimensional structure, the cell is generally observed in a plane, so the term “cell size R” as used herein is based on the diameter of the cell.

In addition to the value of the hardness of the culture portion P which may be adjusted by the area of the tops 111 and the quantity of the protrusions 110, the material of the culture membrane 10 also affects the hardness. When using different materials to produce the culture membranes 10 having the same hardness of the culture portion P, an area of an orthographic projection of the tops 111 of the plurality of protrusions 110 on the first surface 100 as a percentage of an area of the first surface 100 and the spacing D between the centers C of the tops 111 of the plurality of protrusions 110 may also be different. The material of the culture membrane 10 includes, for example, silicone and sapphire, but is not limited thereto.

For example, when the culture portion P has a hardness of 2 kPa to 500 kPa, if a material of the culture membrane 10 has a Shore hardness of less than 60 HA (such as silicone), then the area of the orthographic projection of the tops 111 of the plurality of protrusions 110 on the first surface 100 as a percentage of the area of the first surface 100 is 20% to 70%. In addition, the distance D between the centers C of the tops 111 of the plurality of protrusions 110 is, for example, 0.4 μm to 1 μm.

On the other hand, if a material of the culture membrane 10 has a Shore hardness of more than 60 HA (such as sapphire), then the area of the orthographic projection of the tops 111 of the plurality of protrusions 110 on the first surface 100 as a percentage of the area of the first surface 100 is 0.01% to 0.001%. In addition, the distance D between the centers C of the tops 111 of the plurality of protrusions 110 is, for example, 1 μm to 3 μm.

In the culture membrane 10 using the material having a Shore hardness of less than 60 HA (such as silicone), the area of the orthographic projection of the tops 111 of the plurality of protrusions 110 on the first surface 100 as a percentage of the area of the first surface 100 is 20% to 70%, in other words, the shape of the plurality of protrusions 110 is similar to the cylindrical shape or the truncated cone shape. In the culture membrane 10 using the material having a Shore hardness of more than 60 HA (such as sapphire), the area of the orthographic projection of the tops 111 of the plurality of protrusions 110 on the first surface 100 as a percentage of the area of the first surface 100 is 0.01% to 0.001%. Since the percentage is less than 1%, the shape of the plurality of protrusions 110 is similar to the conical shape.

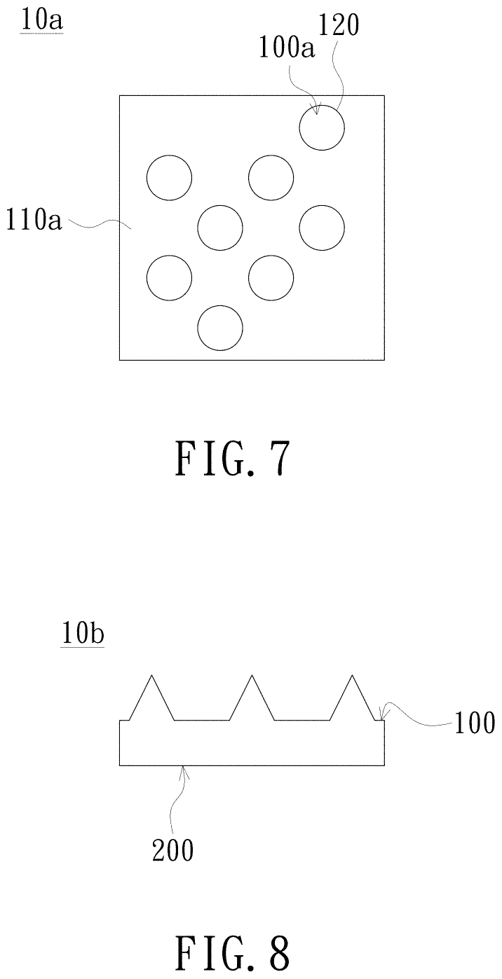

FIG. 7 is a schematic diagram of a culture membrane of another embodiment of the invention. Referring to FIG. 7, the culture membrane 10a of the embodiment is similar in structure and advantages to the culture membrane 10, and only the main differences in the structure will be described below. The culture membrane 10a is made of silicone, wherein the plurality of circles in FIG. 7 is depressions 120. The bottoms 100a of the depressions 120 are the first surface described above, and the surface 110a is the tops of the plurality of protrusions described above (can be seen as the plurality of tops connected to form surface 110a). It should be noted that when the percentage of the area of the orthographic projection of the tops of the plurality of protrusions on the first surface (the area of surface 110a) to the area of the first surface (the area of the bottoms 100a of the depressions 120) is small (such as percentage of sapphire is less than 1%), it is more difficult to present in culture membrane 10a. In other words, this manner is more suitable for use in a culture membrane made of a material having a Shore hardness of less than 60 HA.

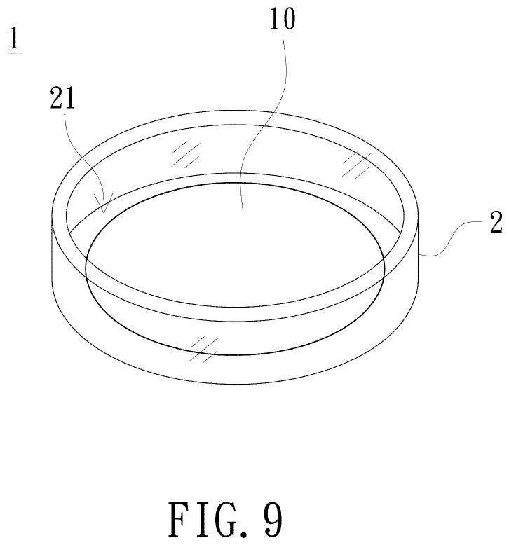

FIG. 8 is a schematic cross-sectional diagram of a culture membrane of another embodiment of the invention. Referring to FIG. 8, the culture membrane 10b of the embodiment is similar in structure and advantages to the culture membrane 10. The culture membrane 10b is made of sapphire and further has a second surface 200 opposite to the first surface 100. The first surface 100 and the second surface 200 are both polished surfaces. Since the sapphire is relatively hard, it is difficult to measure the thickness when it is made into the culture membrane 10b, so it should be polished. The polished first surface 100 and the polished second surface 200 are transparent, so that light is easily transmitted, and cells can be observed more clearly. The first surface 100 and the second surface 200 may be, for example, a high-transparency polished surface, but not limited thereto, the transmittance may be as long as the cells are clearly observed. Another advantage of the culture membrane 10b made of sapphire is that since the sapphire has the characteristics of acid and alkali proof and high temperature resistance, the culture membrane 10b can be reused compared to a conventional plastic culture dish or a glass culture dish, and it does not cause environmental pollution, and may save cost.

The culture membrane 10 may be used in combination with other culture media, in addition to being used alone. FIG. 9 is a schematic perspective diagram of a culture dish of one embodiment of the invention. Referring to FIG. 9, a culture dish 1 of the embodiment includes a dish body 2 and the culture membrane 10. The culture membrane 10 is disposed on a bottom surface 21 of the dish body 2. The function and advantages of the culture dish 1 are the same as those of the culture membrane 10. The culture membrane 10 may be cut into different shapes according to different experimental design requirements, and the circular shape corresponding to the bottom surface 21 is taken as an example in FIG. 9. In addition, the culture membrane 10 may be replaced with the culture membranes 10a, 10b.

In summary, the culture membrane of the invention has a culture portion. Since the culture portion has a hardness of 2 kPa to 500 kPa, which overlaps with the hardness range of the human tissue, the in vivo environment of cell growth may be simulated, and the survival rate of the cultured cells may increase. The culture dish of the invention uses the culture membrane, and therefore has the same advantages.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A culture membrane, having a first surface, wherein the first surface has a plurality of protrusions, the tops of the plurality of protrusions and the gaps between the tops jointly form a culture portion adapted to culture cells, the culture portion has a hardness of 2 kPa to 500 kPa.

2. The culture membrane according to claim 1, wherein the culture portion has a hardness of 2 kPa to 20 kPa.

3. The culture membrane according to claim 1, wherein a shape of the plurality of protrusions includes a conical shape, a cylindrical shape or a truncated cone shape.

4. The culture membrane according to claim 1, wherein a spacing between centers of the tops of the plurality of protrusions is less than or equal to half a size of a cell.

5. The culture membrane according to claim 1, wherein a material of the culture membrane has a Shore hardness of less than 60 HA, and an area of an orthographic projection of the tops of the plurality of protrusions on the first surface as a percentage of an area of the first surface is 20% to 70%.

6. The culture membrane according to claim 5, wherein a distance between centers of the tops of the plurality of protrusions is 0.4 μm to 1 μm.

7. The culture membrane according to claim 5, wherein the material of the culture membrane includes silicone.

8. The culture membrane according to claim 1, further has a second surface opposite to the first surface, and the first surface and the second surface are both polished surfaces, wherein a material of the culture membrane has a Shore hardness of more than 60 HA, and an area of an orthographic projection of the tops of the plurality of protrusions on the first surface as a percentage of an area of the first surface is 0.01% to 0.001%.

9. The culture membrane according to claim 8, wherein a distance between centers of the tops of the plurality of protrusions is 1 μm to 3 μm.

10. The culture membrane according to claim 8, wherein the material of the culture membrane includes sapphire.

11. A culture dish, comprising a dish body and a culture membrane, the culture membrane is disposed on a bottom surface of the dish body, and has a first surface, wherein the first surface has a plurality of protrusions, the tops of the plurality of protrusions and the gaps between the tops jointly form a culture portion adapted to culture cells, the culture portion has a hardness of 2 kPa to 500 kPa.

12. The culture membrane according to claim 11, wherein the culture portion has a hardness of 2 kPa to 20 kPa.

13. The culture membrane according to claim 11, wherein a shape of the plurality of protrusions includes a conical shape, a cylindrical shape or a truncated cone shape.

14. The culture membrane according to claim 11, wherein a spacing between centers of the tops of the plurality of protrusions is less than or equal to half a size of a cell.

15. The culture membrane according to claim 11, wherein a material of the culture membrane has a Shore hardness of less than 60 HA, and an area of an orthographic projection of the tops of the plurality of protrusions on the first surface as a percentage of an area of the first surface is 20% to 70%.

16. The culture membrane according to claim 15, wherein a distance between centers of the tops of the plurality of protrusions is 0.4 μm to 1 μm.

17. The culture membrane according to claim 15, wherein the material of the culture membrane includes silicone.

18. The culture membrane according to claim 11, further has a second surface opposite to the first surface, and the first surface and the second surface are both polished surfaces, wherein a material of the culture membrane has a Shore hardness of more than 60 HA, and an area of an orthographic projection of the tops of the plurality of protrusions on the first surface as a percentage of an area of the first surface is 0.01% to 0.001%.

19. The culture membrane according to claim 18, wherein a distance between centers of the tops of the plurality of protrusions is 1 μm to 3 μm.

20. The culture membrane according to claim 18, wherein the material of the culture membrane includes sapphire.
